(19) United States
(12) Patent Application Publication
(10) Pub. No.: US 2007/0179168 A1
(43) Pub. Date: Aug. 2, 2007

(54) METHODS OF TREATING ANXIETY DISORDERS

(75) Inventors: Michael Cowley, Portland, OR (US); Gary Tollefson, Indianapolis, IN (US)

Correspondence Address: KNOBBE MARTENS OLSON & BEAR LLP, 2040 MAIN STREET, FOURTEENTH FLOOR, IRVINE, CA, 92614, US

(73) Assignee: Orexigen Therapeutics, Inc., San Diego, CA (US)

(21) Appl. No.: 11/563,631

(22) Filed: Nov. 27, 2006

Related U.S. Application Data
(60) Provisional application No. 60/740,000, filed on Nov. 28, 2005.

(51) Int. Cl. A61K 31/485; A61K 31/445; A61K 31/381; A61K 31/137

(52) U.S. Cl. 514/282; 514/317; 514/649; 514/469; 514/447

(57) ABSTRACT

Disclosed are methods of treating an anxiety disorder, e.g., obsessive compulsive disorder, in an individual, comprising identifying an individual in need thereof and treating that individual to antagonize opioid receptor activity and to restore normal monoaminergic tone within the synapse.

### RELATED APPLICATION INFORMATION

This application claims priority to U.S. Provisional Application Ser. No. 60/740,000, filed Nov. 28, 2005, which is hereby incorporated by reference in its entirety.

### BACKGROUND OF THE INVENTION

1. Field of the Invention

The present invention is in the field of methods for the treatment of anxiety disorders, particularly obsessive compulsive disorder, in individuals.

2. Description of the Related Art

Obsessive compulsive disorder afflicts about 3.3 million adult Americans. It strikes men and women in approximately equal numbers and usually first appears in childhood, adolescence, or early adulthood. One-third of adults with OCD report having experienced their first symptoms as children. The course of the disease is variable—symptoms may come and go, they may ease over time, or they can grow progressively worse. Research evidence suggests that OCD might run in families.

Obsessive-Compulsive Disorder, OCD, is an anxiety disorder and is characterized by recurrent, unwanted thoughts (obsessions) and/or repetitive behaviors (compulsions). Repetitive behaviors such as handwashing, counting, checking, or cleaning are often performed with the hope of preventing obsessive thoughts or making them go away. Performing these so-called “rituals,” however, provides only temporary relief, and not performing them markedly increases anxiety.

People with OCD may be plagued by persistent, unwelcome thoughts or images, or by the urgent need to engage in certain rituals. They may be obsessed with germs or dirt, and wash their hands over and over. They may be filled with doubt and feel the need to check things repeatedly.

Obsessions are recurrent, persistent, unwanted ideas, thoughts or impulses that the patient experiences involuntarily and that appear to be senseless. They commonly intrude when the patient is trying to think of or do other things. Common obsessions include: fear of dirt or contamination, concern with order, symmetry and exactness, constantly thinking about certain sounds, images, words or numbers, fear of harming a family member or friend, and fear of thinking evil or sinful thoughts. Compulsions are repetitive behaviors that the patient is driven to perform regularly to combat his or her obsessions, even though to the patient—and to others—they may seem irrational. The patient may even make up “rules” to follow that help control the anxiety he or she feels when having obsessive thoughts. Typical compulsions include: excessive hand washing, repeatedly checking that doors are locked and appliances are turned off, arranging items in a precise order, repeatedly counting to the same number, and touching certain objects a precise number of times. When the patient performs these rituals, he or she may feel some relief from anxiety, but not for long. Soon the patient's discomfort returns, and the patient feels compelled to repeat the behaviors.

Although a definitive cause of obsessive-compulsive disorder has not yet been found, it is considered the product of interactions between biologic predisposition and various developmental and psychosocial influences. Data in the literature support the so-called “serotonin (5-HT) hypothesis” of OCD (Barr et al.): peripheral markers of serotonin function (Bastani et al., 1991), pharmacologic challenge studies with serotonin agonists (Erzegovesi et al., 2001b) and, particularly, drug-response data from selective serotonin reuptake inhibitors (SSRIs) (Greist et al., 1995).

According to the serotonin hypothesis, patients with OCD have a dysregulation in the serotonergic system, with a hypersensitivity of postsynaptic 5-HT receptors, which could account for a different mechanism of action of SSRIs in OCD (than in major depression) (Billett et al., 1997; Zohar et al., 1987). For example, onset of therapeutic action is 10 to 12 weeks in OCD, compared to three to four weeks for mood disorders. SSRI dosing is also often higher than that used in major depression.

At the present time, SSRIs represent the first-line strategy in drug treatment of OCD. However, a major concern in the clinical pharmacology of OCD is the number of non-responders to drug therapy. As many as 40% to 60% of patients treated with SSRIs at full dosages for at least 12 weeks do not show significant improvements in OCD symptoms and their global abilities to function. In particular, the motor or compulsive features of OCD appear to have a limited response to selective serotoninergic drug approaches. Therefore, there is an unmet need in the art for a new line of therapy for OCD patients.

### SUMMARY OF THE INVENTION

In some embodiments, the present invention relates to methods of treating an anxiety disorder comprising identifying a patient suffering from or at risk of suffering from an anxiety disorder, and administering to the patient a first compound and a second compound, wherein said first compound is an opioid antagonist or opioid receptor partial agonist and said second compound modulates monoaminergic synaptic activity. The monoaminergic synaptic activity can be at least one monoaminergic synaptic activity selected from serotonin synaptic activity, norepinephrine synaptic activity, and dopamine synaptic activity. In some embodiments, the anxiety disorder is obsessive compulsive disorder. The first compound and the second compound can be administered nearly simultaneously.

The first compound can be an MOP receptor. In some embodiments, the first compound is selected from alvimopan, norbinaltorphimine, nalmefene, naloxone, naltrexone, methylnaltrexone, and nalorphine, and pharmaceutically acceptable salts, enantiomers, metabolites or prodrugs thereof. The first compound can be naltrexone, 6-beta-naltrexol, or a pharmaceutically acceptable salt or prodrug thereof. The first compound can indirectly modulate a dopamine pathway.

The second compound can be a selective serotonin reuptake inhibitor (SSRI), reuptake promoter, or a specific 5-HT receptor agonist. The SSRI can be selected from fluoxetine, fluvoxamine, sertraline, paroxetine, citalopram, escitalopram, sibutramine, duloxetine, and venlafaxine, and pharmaceutically acceptable salts or prodrugs thereof. In some embodiments, the SSRI is fluoxetine or a pharmaceutically acceptable salt or prodrug thereof. In other embodiments, the second compound is bupropion.

In some embodiments, the first compound is naltrexone and the second compound is fluoxetine. In other embodiments, the first compound is naltrexone and the second compound is bupropion

In some embodiments, the present invention relates to a method of treating a movement disorder comprising identifying a patient suffering from or at risk of suffering from a movement disorder, and administering to the patient a first compound and a second compound, wherein said first compound is an opioid antagonist or opioid receptor partial agonist and said second compound modulates monoaminergic synaptic activity. The monoaminergic synaptic activity can be at least one monoaminergic synaptic activity selected from serotonin synaptic activity, norepinephrine synaptic activity, and dopamine synaptic activity. The movement disorder can be Tourette syndrome or tics.

The first compound can be an MOP receptor antagonist. The second compound can be a selective serotonin reuptake inhibitor (SSRI) or a specific 5-HT receptor agonist. In some embodiments, first compound is naltrexone and the second compound is fluoxetine. In other embodiments, first compound is naltrexone and the second compound is bupropion.

### DETAILED DESCRIPTION OF THE PREFERRED EMBODIMENTS

In a first aspect, the present invention is directed to a method of treating an anxiety disorder comprising identifying an individual in need thereof, and administering to the individual a first compound and a second compound, where the first compound is a functional opioid antagonist and the second compound modulates serotonergic receptor tone. In preferred embodiments, the anxiety disorder is obsessive compulsive disorder (OCD). In some embodiments, the anxiety disorder is treated by identifying a patient suffering from an anxiety disorder and administering to the patient naltrexone or a related enantiomer, metabolite, or prodrug and an antidepressant medication e.g., bupropion, or enantiomers, metabolites, or prodrugs thereof. In other embodiments, the anxiety disorder is treated by identifying a patient suffering from an anxiety disorder and administering to the patient naltrexone and a selective serotonin acting compound e.g., fluoxetine, or enantiomers, metabolites, or prodrugs thereof.

In a second aspect, the present invention is directed to a method of treating a movement disorder including compulsive or ritualistic behaviors comprising identification of an individual in need thereof, and administering to the individual a first compound and a second compound, where the first compound is an opioid antagonist and the second compound causes increased agonism at monamine receptors including dopamine and serotonin. In some embodiments, the movement disorder is treated by identifying a patient suffering from an anxiety disorder and administering to the patient naltrexone and a modest dopamine enhancer, such as bupropion, or enantiomers, metabolites, or prodrugs thereof. In other embodiments, the movement disorder is treated by identifying a patient suffering from an anxiety disorder and administering to the patient naltrexone and a serotoninergic enhancer such as fluoxetine, or enantiomers, metabolites or prodrugs thereof.

Definitions

The term “pharmaceutically acceptable salt” refers to a formulation of a compound that does not cause significant irritation to an organism to which it is administered and does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl) methylamine, and salts thereof with amino acids such as arginine, lysine, and the like.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug, or may demonstrate increased palatability or be easier to formulate. An example, without limitation, of a prodrug would be a compound of the present invention which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to provide the active moiety.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The terms “serotonin 1A”, “serotonin 1B receptor,” “serotonin 2C receptor,” “5-HT1b receptor,” and “5-HT2c receptor” refer to receptors found more commonly in rodents. It is understood by those of skill in the art that other mammals have serotonin receptors on various neurons that are analogous in function and form to these receptors. Agonists, antagonists, or synaptic re-uptake inhibitors at these non-rodent, preferably human, serotonin receptors are within the scope of the present invention.

A compound that modulates monoaminergic synaptic activity, (e.g. serotonin, norepinephrine, dopamine, etc.), is any compound that increases or decreases the synaptic activity at a monoaminergic synapse in one or more brain regions, (e.g., the limbic system or frontal cortex (e.g., orbito-lateral pre-frontal region)). Synaptic activity can be modulated by increasing or decreasing any characteristic of synaptic activity, for example the intensity or duration. These compounds include, for example receptor agonists, antagonists, inverse agonists, or partial agonists, as well as reuptake inhibitors. Also included are compounds with mixed affinity for more than one monoaminergic pathway, e.g. a mixed dopamine/norepinephrine reuptake inhibitor.

The term “treating” or “treatment” does not necessarily mean total cure. Any alleviation of any undesired signs or symptoms of the disease to any extent or the slowing down of the progress of the disease can be considered treatment. Furthermore, treatment may include acts that may worsen the patient's overall feeling of well being or appearance. Treatment may also include lengthening the life of the patient, even if the symptoms are not alleviated, the disease conditions are not ameliorated, or the patient's overall feeling of well being is not improved. Thus, in the context of the present invention, reducing the obsessive feelings or reducing the frequency or intensity of compulsive behavior may be considered treatment, even if the patient is not cured or does not generally feel better, or even feel worse due to certain side effects of the medication.

“Anxiety” includes without limitation the unpleasant emotional state comprising psychophysiological responses to anticipation of unreal or imagined danger, ostensibly resulting from unrecognized intrapsychic conflict. Physiological concomitants include increased heart rate, altered respiration rate, sweating, trembling, weakness, and fatigue; psychological concomitants include feelings of impending danger, powerlessness, apprehension, and tension. See Dorland's Illustrated Medical Dictionary, W. B. Saunders Co., 27th ed. (1988).

“Anxiety Disorder” includes without limitation mental disorders in which anxiety and avoidance behavior predominate. Examples of anxiety disorders include obsessive-compulsive disorder, post traumatic stress disorder, social anxiety, somatization, specific social phobia, premenstrual dysphoric dosrder, anxiety associated with a medical condition, adjustment disorder, dysthymia, specific phobia, fibromyalgia, panic attacks, agoraphobia, acute stress disorder, a generalized anxiety disorder, and/or a substance-induced anxiety disorder. Other anxiety disorders are characterized in Diagnostic and Statistical Manual of Mental Disorders (American Psychiatric Association 4th ed. 1994). The skilled artisan will recognize that there are alternative nomenclatures, nosologies, and classification systems for pathologic psychological conditions and that these systems evolve with medical scientific progress. In preferred embodiments, methods, compounds, and compositions described herein to treat an anxiety disorder can be used to treat obsessive compulsive disorder.

“Obsessive compulsive disorder” or “OCD” is an anxiety disorder characterized by recurrent obsessions or compulsions sufficient to cause marked distress in the individual. They are typically time-consuming, and/or significantly interfere with the person's normal functioning, social activities, or relationships. Obsessions are recurrent ideas, thoughts, images, or impulses that enter the mind and are persistent, intrusive, and unwelcome. Often, attempts are made to ignore or suppress the thoughts, or to neutralize them with some other thought or action. The individual may recognize the obsessions as a product of his or her own mind. Compulsions are repetitive, purposeful behaviors or movements performed in response to an obsession, and are typically designed to neutralize or prevent discomfort or some dreaded event or situation. For example, a common obsession concerns thoughts of contamination; excessive, repetitive, and non-purposeful handwashing is a common compulsion.

“Movement disorder”, as used herein, is used to refer to all forms of abnormal and involuntary movements, including vocalizations. Movement disorders include, for example, tardive dyskinesia (TD), tics, Gilles de la Tourette syndrome (TS), Parkinson's disease, Huntington's disease, and focal dystonias such as blepharospasm.

Movement Disorders

There is also a strong association of obsessive compulsive disorder with movement disorders. Obsessive compulsive disorder is associated with Gilles de la Tourette syndrome (Tourette syndrome), as well as with several other basal ganglia diseases including Sydenham's chorea and Huntington's disease. There is strong evidence of a link between obsessive compulsive disorder and motor tics. While estimates of the occurrence of obsessive compulsive disorder in patients with Tourette syndrome vary from 5% to over 50%, all estimates are significantly higher than the prevalence of obsessive compulsive disorder in the general population. Shared clinical features between obsessive compulsive disorder and Tourette syndrome include “waxing and waning of symptoms, early age at onset, ego-dystonic behavior (i.e., behavior contrary to an individuals conscious preferences), worsening with depression and anxiety, and their occurrence in the same families” (Robertson and Yakely, supra). Genetic studies suggest that in some families, there is a single autosomal dominant gene that can be expressed phenotypically as Tourette syndrome, obsessive compulsive disorder, or both. Tourette syndrome is most often treated with dopamine antagonists and obsessive compulsive disorder with serotonin reuptake inhibitors. However, the addition of dopamine antagonists can augment the therapeutic efficacy of serotonin reuptake inhibitors in obsessive compulsive disorder, and the addition of serotonin reuptake inhibitors can augment the efficacy of dopamine antagonists in Tourette syndrome. All of these considerations support the idea that there are overlapping physiologic mechanisms for obsessive compulsive disorder and Tourette syndrome.

Both tics and obsessive compulsive disorder can be produced by the CNS effects of an autoimmune reaction to infection with Group A beta-hemolytic streptococcus—the PANDAS syndrome—Pediatric AutoImmune Disorders Associated with Streptococcus. (Swedo S E, et al: Pediatric autoimmune neuropsychiatric disorders associated with streptococcal infections: clinical description of the first 50 cases. Am J Psychiatry, 155:264-71, February 1998). Similarly, traumatic brain injury can lead to the simultaneous new onset of tics and obsessive compulsive disorder symptoms (Krauss J K; Jankovic J: Tics secondary to craniocerebral trauma. Mov Disord, 12:776-82, September 1997).

Obsessive compulsive disorder symptoms were compared between patients with blepharospasm, a focal dystonia caused by basal ganglia dysfunction, and hemifacial spasm, a syndrome with superficially similar symptoms but due to peripheral nerve dysfunction. The blepharospasm patients had significantly more obsessive compulsive disorder symptoms on a symptom check list (Broocks, et al.: Higher prevalence of obsessive-compulsive compulsive symptoms in patients with blepharospasm than in patients with hemifacial spasm. Am J Psychiatry, 155:555-7, April 1998).

Obsessive compulsive disorder is not only associated with Tourette syndrome, but obsessive-compulsive phenomena share common clinical features with tics. Both involve repetitive, stereotyped, involuntary phenomena. In the case of obsessive compulsive disorder, these are thoughts or purposeful motor sequences (compulsive rituals); in the case of tics they are simpler, non-purposeful movements. Both involve activation of neocortical representations by limbic or striatal inputs.

Therefore, embodiments of this invention also include compounds, compositions and methods to treat movement disorders.

First Compound

In certain embodiments the first compound of compositions and methods described herein is an opioid antagonist, and in some of these embodiments it antagonizes a μ-opioid receptor (MOP-R) and/or a kappa-opioid receptor (KOP-R) in a mammal. In some embodiments the opioid antagonist is selected from the group consisting of alvimopan, norbinaltorphimine, nalmefene, naloxone, naltrexone, methylnaltrexone, 6-β-hydroxynaltrexone, and nalorphine, and pharmaceutically acceptable salts or prodrugs thereof. In some preferred embodiments, the opioid partial agonist or antagonist is naltrexone, administered as either an immediate release or sustained (SR) release drug formulation.

In other embodiments, the opioid antagonist is selectively a partial opioid agonist or pure antagonist. Compounds of this class may have some agonist activity at opioid receptors. However, because they are weak agonists, they function as de-facto antagonists. Examples of partial opioid agonists include pentacozine, buprenorphine, nalorphine, propiram, and lofexidine. Example of a pure or selective antagonist is 6-beta-naltrexol.

Second Compound

The second compound may increase the extracellular monoaminergic levels in various brain regions e.g., the limbic system or frontal cortex (e.g., orbito-lateral pre-frontal region). Relevant monoamines or neurotransmitters in these regions of interest include serotonin, dopamine, norepinephrine, gamma-amino-butryic acid (GABA), and glutamate. In some embodiments, the second compound is selected from the group consisting of a selective serotonin reuptake inhibitor (SSRI), a serotonin 1A agonist/partial agonist/reverse agonist/or antagonist, a serotonin 2C agonist, and/or a serotonin 1B agonist. In further embodiments, the second compound is selected, e.g., from the group consisting of fluoxetine, fluvoxamine, sertraline, paroxetine, citalopram, escitalopram, sibutramine, duloxetine, venlafaxine, sumatriptan, almotriptan, naratriptan, frovatriptan, rizatriptan, zomitriptan, and elitriptan, and pharmaceutically acceptable salts, enatiomers, metabolites, or prodrugs thereof. In some preferred embodiments, the second compound is fluoxetine.

In certain embodiments, the second compound suppresses the expression of the brain-derived neurotrophic factor (BDNF) gene or the production or release of a neuropeptide e.g., oxytocin or vasopressin In some of these embodiments, the second compound suppresses the activity of neurons that express vasopressin.

In other embodiments, the second compound suppresses the expression of the NPY gene or the production or release of neuropeptide Y (NPY). In some of these embodiments, the second compound suppresses the activity of neurons that express NPY. In further embodiments, the second compound is selected from the group consisting of NPY antagonists, ghrelin antagonists, and leptin. In certain other embodiments, the second compound antagonizes NPY Y1 or Y2 receptors.

Other embodiments of the present invention include those in which the second compound is selected from the group consisting of a γ-amino butyric acid (GABA) inhibitor, a GABA receptor antagonist, and a GABA channel modulator. By “GABA inhibitor” it is meant a compound that reduces the production of GABA in the cells, reduces the release of GABA from the cells, or reduces the activity of GABA on its receptors, either by preventing the binding of GABA to GABA receptors or by minimizing the effect of such binding. The GABA inhibitor may be a 5-HT1b agonist or another agent that inhibits the activity of NPY/AgRP/GABA neurons. In addition, the GABA inhibitor may suppress the expression of the AgRP gene, or the GABA inhibitor may suppress the production or release of AgRP. It is, however, understood that a 5-HT1b agonist may inhibit the NPY/AgRP/GABA neuron (and therefore activate pro-opiomelanocortin (POMC) neurons) without acting as an inhibitor of the GABA pathway.

In certain other embodiments the GABA inhibitor increases the expression of the POMC gene. In some of these embodiments, the GABA inhibitor increases the production or release of POMC protein. In certain other of these embodiments, the GABA inhibitor increases the activity on POMC expressing neurons. In some embodiments, the GABA inhibitor is topiramate.

In other embodiments the second compound is a dopamine reuptake inhibitor or receptor antagonist. Phentermine is an example of a dopamine reuptake inhibitor. Haloperidol, ocaperidone, risperidone, olanzapine, quetiapine, amisulpride, and pimozide are examples of dopamine receptor antagonists. In certain other embodiments, the second compound is a norepinephrine reuptake inhibitor. Examples of norepinephrine reuptake inhibitors include bupropion, thionisoxetine, atomoxetine, and reboxetine. Other embodiments include those in which the second compound is a dopamine agonist. Some dopamine agonists that are available on the market include cabergoline, amantadine, lisuride, pergolide, ropinirole, pramipexole, and bromocriptine.

In some embodiments, the second compound is bupropion. In other embodiments, the second compound is a metabolite or enantiomer of bupropion. The metabolites of bupropion suitable for inclusion in the methods, compounds, and compositions disclosed herein include the erythro- and threo-amino alcohols of bupropion, the erythro-amino diol of bupropion, and morpholinol metabolites of bupropion. In some embodiments, the metabolite of bupropion is (±)-(2R*,3R*)-2-(3-chlorophenyl)-3,5,5-trimethyl-2-morpholinol. In some embodiments the metabolite is (−)-(2R*,3R*)-2-(3-chlorophenyl)-3,5,5-trimethyl-2-morpholinol, while in other embodiments, the metabolite is (+)-(2S,3S)-2-(3-chlorophenyl)-3,5,5-trimethyl-2-morpholinol. Preferably, the metabolite of bupropion is (+)-(2S,3S)-2-(3-chlorophenyl)-3,5,5-trimethyl-2-morpholinol, which is known by its common name of radafaxine, which is described in U.S. Pat. No. 6,274,579, issued on Aug. 14, 2001 to Morgan et al., which is hereby incorporated by reference herein in its entirety, including any drawings.

In further embodiments, the second compound is an anticonvulsant. The anticonvulsant may be selected from the group consisting of zonisamide, topiramate, nembutal, alprazolam, diazepam, lorazepam, clonazepam, clorazepate, tiagabine, gabapentin, fosphenytoin, phenytoin, carbamazepine, valproate, felbamate, levetiracetam, oxcarbazepine, lamotrigine, methsuximide, and ethosuxmide.

In certain embodiments, the second compound itself may be a combination of two or more compounds. For example, the second compound may be a combination of a dopamine reuptake inhibitor and a norepinephrine reuptake inhibitor, e.g. bupropion, atomoxetine, reboxetine, and mazindol. Alternatively, the second compound may be a combination of a SSRI and a norepinephrine reuptake inhibitor, such as sibutramine, milnacipran, venlafaxine, and duloxetine.

In certain embodiments, the second compound is an activator of the POMC neurons. Examples of POMC activators include Ptx1 and interleukin 1 beta, (IL-1β). Alternatively the second compound may enhance reuptake of serotonin and/or norepinephrine such a tianeptine or amineptine.

Compound Combinations

In some embodiments, the following combinations of compounds are administered or comprised in a composition:
a SSRI in combination with a dopamine reuptake inhibitor, a dopamine/norepinephrine reuptake inhibitor, a norepinephrine reuptake inhibitor, an opioid antagonist, a partial opioid agonist, GABA inhibitor, or a peptide, such as PYY, PYY₃₋₃₆, or leptin;
Serotonin in combination with a dopamine reuptake inhibitor, a dopamine/norepinephrine reuptake inhibitor, an opioid antagonist, a partial opioid agonist, or a GABA inhibitor;
a dopamine reuptake inhibitor in combination with a norepinephrine reuptake inhibitor, a norepinephrine releaser, a norepinephrine agonist, an opioid antagonist, a partial opioid agonist, a GABA inhibitor, an adenosine compound, a cholinergic receptor antagonist, or a peptide, such as PYY, PYY3-36, or leptin;
a dopamine/norepinephrine reuptake inhibitor in combination with an opioid antagonist, a partial opioid agonist, or a GABA inhibitor;
naltrexone and bupropion;
naltrexone and fluoxetine;
a dopamine agonist in combination with an opioid antagonist, a partial opioid agonist, a GABA inhibitor, or a peptide, such as PYY, PYY3-36, or leptin;
a serotonin and/or norepinephrine reuptake enhancer with an opioid antagonist e.g., tianeptine or it's metabolites/enantiomers/or prodrugs combined with naltrexone or it's meatbolites/enantiomers/or pro-drugs.

Examples of norepinephrine agonists include phendimetrazine and benzphetamine. Examples of adenosine compounds include all xanthine derivatives, such as adenosine, caffeine, theophylline, theobromine, and aminophylline. An example of a nicotinic cholinergic receptor agonist is nicotine, and muscarinic cholinergic agonist is xanomeline.

Patient Identification

First and second compounds described herein can be administered to a patient suffering or at risk of suffering from an anxiety disorder. In preferred embodiments, the anxiety disorder is OCD. A patient can be at risk of suffering from an anxiety disorder if the patient previously experienced an anxiety disorder, is suffering from another condition (such as depressive or movement disorders) which increases the probability of experiencing an anxiety disorder, or is genetically predisposed to an anxiety disorder. First and second compounds described herein can be administered to a patient suffering or at risk of suffering from a movement disorder. In some preferred embodiments, the movement disorder is tics. In other preferred embodiments, the movement disorder is Tourette syndrome. A patient can be at risk of suffering from a movement disorder if the patient previously experienced a movement disorder, is suffering from another condition (such as an anxiety disorder) which increases the probability of experiencing a movement disorder, or is genetically predisposed to a movement disorder.

In some embodiments, first and second compounds described herein can be administered to a patient suffering or at risk of suffering from both a movement disorder and an anxiety disorder. In other embodiments, the patient may be suffering from an anxiety disorder and at risk of suffering from a movement disorder. In still other embodiments, the patient may be suffering from a movement disorder and at risk of suffering from an anxiety disorder. The patient may also be suffering from an anxiety or a movement disorder and not be at risk of suffering from the other disorder.

The patient may be selected from the group consisting of mice; rats; rabbits; guinea pigs; dogs; cats; sheep; goats; cows; primates, such as monkeys, chimpanzees, and apes; and humans.

Individuals suffering from depression may also have an anxiety disorder, specifically OCD. In certain embodiments, the patient is not suffering from depression.

Administration

In some embodiments, patients are administered a first and second compound as described herein, wherein the first compound and the second compound nearly simultaneously. These embodiments include those in which the two compounds are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc. In some embodiments, a patient is infused with an intravenous formulation of one compound prior to the infusion of an intravenous formulation of the other compound. In these embodiments, the infusion may take some time, such as a few minutes, a half hour, or an hour, or longer. If the two intravenous infusions are done one right after the other, such administration is considered to be nearly simultaneously within the scope of the present disclosure, even though there was a lapse of some time between the start of one infusion and the start of the next infusion.

In other embodiments the administering step comprises administering one of the first compound and the second compound first and then administering the other one of the first compound and the second compound. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally. In further embodiments, the patient may receive both compounds on a routine or continuous basis, such a continuous infusion of the compound through an IV line.

In certain embodiments, the first compound and the second compound are administered individually. In other embodiments, the first compound and the second compound are covalently linked to each other such that they form a single chemical entity. The single chemical entity is then digested and is metabolized into two separate physiologically active chemical entities, one of which is the first compound and the other one is the second compound.

In certain embodiments disclosed herein, an individual is given a pharmaceutical composition comprising a combination of two or more compounds to treat an anxiety and/or movement disorder. In some of these embodiments, each compound is a separate chemical entity. However, in other embodiments, the two compounds are joined together by a chemical linkage, such as a covalent bond, so that the two different compounds form separate parts of the same molecule. The chemical linkage is selected such that after entry into the body, the linkage is broken, such as by enzymatic action, acid hydrolysis, base hydrolysis, or the like, and the two separate compounds are then formed.

Routes of Administration

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions and/or compounds of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions and/or compounds for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions and/or compounds may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compounds may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The exact formulation and route of administration for the pharmaceutical compositions of the present invention can be chosen by the individual physician in view of the patient's condition. (See e.g., Fingl et al. 1975, in “The Pharmacological Basis of Therapeutics”, Ch. 1 p. 1).

Pharmaceutical Carriers

In another aspect, the invention relates to a pharmaceutical composition comprising a combination of an opioid antagonist and a compound that modulates monoaminergic systems as compared to normal physiological conditions, as described above, or comprising a linked molecule, as described herein, and a physiologically acceptable carrier, diluent, or excipient, or a combination thereof.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g, polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms. One or more of the compounds described herein can be formulated as a controlled release form, e.g., as a sustained release form.

Dosages

Pharmaceutical compositions and/or compounds suitable for use in the present invention include compositions and/or compounds where the active ingredients are contained in an amount effective to achieve its intended purpose. More specifically, a therapeutically effective amount means an amount of compound effective to inhibit, delay, prevent, alleviate or ameliorate symptoms of disease or prolong the survival of the subject being treated. Determination of a therapeutically effective amount is well within the capability of those skilled in the art, especially in light of the detailed disclosure provided herein.

The exact dosage for the pharmaceutical compositions and/or compounds of the present invention can be chosen by the individual physician in view of the patient's condition. (See e.g., Fingl et al. 1975, in “The Pharmacological Basis of Therapeutics”, Ch. 1 p. 1). Typically, the dose range of the composition and/or compound administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the compounds or compositions of the present invention or a pharmaceutically acceptable salt thereof calculated as the free base, the compounds or compositions being administered 1 to 4 times per day. Alternatively the compounds or compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma and/or central nervous system levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition or compounds administered will, of course, be dependent on the subject being treated, on the subject's weight, age, concurrent medical status, use of concomitant drugs, ethnicity, or gender, the severity of the affliction, the manner of administration and the judgment of the prescribing physician.

The compositions or compounds may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions and/or compounds comprising a compound of the invention formulated in a compatible pharmaceutical carrier may also be prepared, placed in an appropriate container, and labeled for treatment of an indicated condition.

It will be understood by those of skill in the art that numerous and various modifications can be made without departing from the spirit of the present invention. Therefore, it should be clearly understood that the forms of the present invention are illustrative only and are not intended to limit the scope of the present invention. Any of the compositions disclosed in any of the embodiments disclosed herein (including the following examples) can be used in the preparation of a medicament for the treatment of an anxiety or movement disorder, as described herein.

### EXAMPLES

The examples below are non-limiting and are merely representative of various aspects of the invention.

### Example 1

### Combination of Fluoxetine and Naltrexone/Naltrexone Metabolites

Individuals having obsessive compulsive disorder are identified. Each individual is instructed to take one 20 mg tablet of fluoxetine (PROZAC®) on a daily basis, in addition to one 50 mg tablet of naltrexone on a daily basis.

The individuals are monitored for a period of months and their behavior is recorded. If the initial dosage is not effective, then the fluoxetine dosage can be increased by 20 mg per day, though rarely exceeding 80 mg total per day.

Fluoxetine has a physiological half life of about 24 hours, whereas that of naltrexone is about 1.5 hours. However their metabolites may demonstrate half-lives in excess of 24 hours. Thus, in some cases, it may be beneficial to administer one dose of fluoxetine per day in conjunction with two or three or more doses of naltrexone throughout the day. Naltrexone may also be in a time-release formulation where the dose is administered once a day, but naltrexone gradually enters the blood stream throughout the day, or in the course of a 12 hour period.

Symptoms of the obsessive compulsive disorders are inhibited in individuals being administered fluoxetine and naltrexone. Adverse events associated with the obsessive compulsive disorders are reduced in individuals being administered fluoxetine and naltrexone. The effects of administration of both fluoxetine and naltrexone on obsessive compulsive disorder are synergistic compared to effects of those expected by administration of fluoxetine and naltrexone alone.

### Example 2

### Combination of Fluoxetine and Nalmefene

Individuals having obsessive compulsive disorder are identified. Each individual is instructed to take one 20 mg tablet of fluoxetine (PROZAC®) on a daily basis. In addition, each individual is injected with 1 mL of a solution of 100 μg of nalmefene in 1 mL of saline, intravenously, intramuscularly, or subcutaneously.

The individuals are monitored for a period of months and their behavior is recorded. If the initial dosage is not effective, then the fluoxetine dosage can be increased by 20 mg per day, though never exceeding 80 mg total per day. In addition, the dosage of nalmefene may be increased up to 2 mL of a solution of 1 mg of nalmefene in 1 mL of saline.

Symptoms of the obsessive compulsive disorders are inhibited in individuals being administered fluoxetine and nalmefene. Adverse events associated with the obsessive compulsive disorders are reduced in individuals being administered fluoxetine and nalmefene. The effects of administration of both fluoxetine and nalmefene on obsessive compulsive disorder are synergistic compared to effects of those expected by administration of fluoxetine and nalmefene alone.

### Example 3

### Combination of Fluoxetine and Naloxone

Individuals having obsessive compulsive disorder are identified. Each individual is instructed to take one 20 mg tablet of fluoxetine (PROZAC®) on a daily basis. In addition, each individual is injected with 1 mL of a solution of 400 μg of naloxone in 1 mL of saline, intravenously, intramuscularly, or subcutaneously.

The individuals are monitored for a period of months and their behavior is recorded. If the initial dosage is not effective, then the fluoxetine dosage can be increased by 20 mg per day, though never exceeding 80 mg total per day.

Symptoms of the obsessive compulsive disorders are inhibited in individuals being administered fluoxetine and naloxone. Adverse events associated with the obsessive compulsive disorders are reduced in individuals being administered fluoxetine and naloxone. The effects of administration of both fluoxetine and naloxone on obsessive compulsive disorder are synergistic compared to effects of those expected by administration of fluoxetine and naloxone alone.

### Example 4

### Combination of Opioid Antagonist and Bupropion

Individuals having obsessive compulsive disorder are identified. Each individual is instructed to take nalmefene, naltrexone, or naloxone in the dosage set forth in Examples 1-3. In addition, each individual is instructed to take bupropion. The usual adult does is 300 mg per day, given three times daily. Dosing should begin at 200 mg per day, given as 100 mg twice daily. Based on clinical response, this dose may be increased to 300 mg per day, given as 100 mg three times daily. No single dose is to exceed 150 mg.

The individuals are monitored for a period of months and their behavior is recorded.

Symptoms of the obsessive compulsive disorders are inhibited in individuals being administered bupropion and naltrexone, nalmefene, or naloxone. Adverse events associated with the obsessive compulsive disorders are reduced in individuals being administered bupropion and naltrexone, nalmefene, or naloxone. The effects of administration of both bupropion and naltrexone, nalmefene, or naloxone on obsessive compulsive disorder are synergistic compared to effects of those expected by administration of bupropion and naltrexone, nalmefene, or naloxone alone.

What is claimed is:

1. A method of treating an anxiety disorder comprising identifying a patient suffering from or at risk of suffering from an anxiety disorder, and administering to the patient a first compound and a second compound, wherein said first compound is an opioid antagonist or opioid receptor partial agonist and said second compound modulates monoaminergic synaptic activity.

2. The method of claim 1, wherein the monoaminergic synaptic activity is at least one monoaminergic synaptic activity selected from serotonin synaptic activity, norepinephrine synaptic activity, and dopamine synaptic activity.

3. The method of claim 1, wherein the anxiety disorder is obsessive compulsive disorder.

4. The method of claim 1, wherein the first compound is a MOP receptor antagonist.

5. The method of claim 1, wherein the first compound is selected from alvimopan, norbinaltorphimine, nalmefene, naloxone, naltrexone, methylnaltrexone, and nalorphine, and pharmaceutically acceptable salts, enantiomers, metabolites or prodrugs thereof.

6. The method of claim 1, wherein the first compound is naltrexone, 6-beta-naltrexol, or a pharmaceutically acceptable salt or prodrug thereof.

7. The method of claim 6, wherein the first compound indirectly modulates a dopamine pathway.

8. The method of claim 1, wherein said second compound is a selective serotonin reuptake inhibitor (SSRI), reuptake promoter, or a specific 5-HT receptor agonist.

9. The method of claim 8, wherein said SSRI is selected from fluoxetine, fluvoxamine, sertraline, paroxetine, citalopram, escitalopram, sibutramine, duloxetine, and venlafaxine, and pharmaceutically acceptable salts or prodrugs thereof.

10. The method of claim 8, wherein said SSRI is fluoxetine or a pharmaceutically acceptable salt or prodrug thereof.

11. The method of claim 1, wherein said first compound and said second compound are administered nearly simultaneously.

12. The method of claim 1, wherein the second compound is bupropion or a metabolite thereof.

13. The method of claim 1, wherein said first compound is naltrexone and said second compound is fluoxetine.

14. The method of claim 1, wherein said first compound is naltrexone and said second compound is bupropion.

15. A method of treating a movement disorder comprising identifying a patient suffering from or at risk of suffering from a movement disorder, and administering to the patient a first compound and a second compound, wherein said first compound is an opioid antagonist or opioid receptor partial agonist and said second compound modulates monoaminergic synaptic activity.

16. The method of claim 15, wherein the monoaminergic synaptic activity is at least one monoaminergic synaptic activity selected from serotonin synaptic activity, norepinephrine synaptic activity, and dopamine synaptic activity.

17. The method of claim 15, wherein the movement disorder is Tourette syndrome.

18. The method of claim 15, wherein the movement disorder is tics.

19. The method of claim 15, wherein the first compound is a MOP receptor antagonist.

20. The method of claim 15, wherein said second compound is a selective serotonin reuptake inhibitor (SSRI) or a specific 5-HT receptor agonist.

21. The method of claim 15, wherein said first compound is naltrexone and said second compound is fluoxetine.

22. The method of claim 15, wherein said first compound is naltrexone and said second compound is bupropion.
